(19) United States
(12) Patent Application Publication
(10) Pub. No.: US 2008/0293812 A1
(43) Pub. Date: Nov. 27, 2008

(54) Use of pamoic acid or one of its derivatives, or one of its analogues, for the preparation of a medicament for the treatment of diseases characterised by deposits of amyloid aggregates

(75) Inventors: Maria Grazia Gallo, Pomezia (Rome) (IT); Maria Grazia Cima, Pomezia (Rome) (IT); Fabrizio Giorgi, Pomezia (Rome) (IT); Maria Ornella Tinti, Pomezia (Rome) (IT); Paola Piovesan, Pomezia (Rome) (IT); Orlando Ghirardi, Pomezia (Rome) (IT)

Correspondence Address: NIXON & VANDERHYE, PC, 901 NORTH GLEBE ROAD, 11TH FLOOR, ARLINGTON, VA, 22203, US

(73) Assignee: Sigma-Tau Industrie Farmaceutiche Riunite S. P.A.,, Rome (IT)

(21) Appl. No.: 11/979,301

(22) Filed: Nov. 1, 2007

Related U.S. Application Data
(62) Division of application No. 10/311,888, filed on Jan. 31, 2003, now abandoned.

(30) Foreign Application Priority Data
Jun. 23, 2000 (IT) RM2000A000340

(51) Int. Cl. A61K 31/216; C07C 69/76; A61P 25/28

(52) U.S. Cl. 514/548; 560/139

(57) ABSTRACT

Pamoic acid or its derivatives of formula (I) are used to treat diseases characterized by deposits of amyloid aggregates.

The invention described herein relates to the use of pamoic acid or one of its derivatives, or one of its analogues, or one of the pharmaceutically acceptable salts of these, for the preparation of a medicament for the treatment of diseases characterised by deposits of amyloid aggregates.

The presence of amyloid deposits and of abnormalities of the neuronal cytoskeleton are among the most marked manifestations of Alzheimer's disease (AD). These two events, which mainly affect the cerebral cortex at an early stage, even though the final pathological picture of the disease involves the entire central nervous system, are a necessary though not in themselves sufficient condition for onset of the disease (Chen M. (1998) Frontiers in Bioscience 3a, 32-37).

In general, regardless of the protein from which it is formed, the substance amyloid has the characteristics of being composed of fibres measuring 7-8 nm in diameter, of having affinity for Congo Red and not being soluble in water. In AD, amyloid fibres accumulate external to the cell, in the cerebral intracellular spaces and in the tunica media of the cortical and meningeal arterioles, leading to the formation of three different macroscopic abnormalities: the senile plaques and the diffuse plaques, which differ according to the presence or otherwise of an abnormality of the neuronal processes around the central amyloid deposit, and amyloid angiopathy, which is an expression of infiltration of amyloid fibres into the walls of the arteries, between the smooth muscle fibres and the internal elastic lamina.

Apart from the formation of amyloid and helical filaments, a very serious synaptic rarefaction has been detected in the cortex of subjects suffering from AD. Approximately 80%-90% of neuronal contacts are destroyed in the final phase, of the disease and this abnormality is the actual pathological correlate of dementia. On analysing the dementia trend, it would appear certain that amyloid is the early, primary abnormality of the disease and that the intraneuronal helical filaments are an intermediate expression of the distress of the neurons, which eventually lose their synaptic contacts, with the resulting clinical effect of a deterioration of mental functions.

The soluble form of a particular type of β amyloid, βA₁₋₄₂, so far regarded as toxic only in its aggregate form, is involved in the progressive loss of memory and cognitive functions of Alzheimer's patients. βA₁₋₄₂, which is produced in the initial phase of the disease, suppresses the activity of pyruvate dehydrogenase which fuels the synthesis of ACh providing for the transport of acetyl-CoA, reducing the release of the neurotransmitter, modifying the synaptic connections and causing the cholinergic deficits responsible for the disease (Hoshi M., Takashima A., Murayama M., Yasutake K., Yoshida N., Ishiguro K., Hoshino T., Imahori K. (1997) The Journal of Biological Chemistry 272:4, 2038-2041).

It is known that a number of dyes bind to amyloid fibres in a specific manner and the most important of these is Congo Red (CR) (Lorenzo A. and Yankner B. A, 1994 PNAS 91; 12243-12247).

This dye causes an increase in birefringence of the amyloid fibres and gives rise to a characteristic circular dichroism indicative of a specific interaction between the dye and the substrate (the fibres) facilitating the diagnostic detection of amyloidosis in the tissue.

The β-amyloid protein (βA) derives from the proteolytic action of a number of specific enzymes on the precursor of the amyloid protein (βAPP) (Vassar R. et al. 1999 Science 286; 735-740).

The mechanisms whereby the β-amyloid fragment may induce neurotoxic effects are multiple. In the first place, immunohistochemical studies have revealed the presence, in senile plaques, of inflammatory interleukins (IL-1, IL-6), complement factors, other inflammatory factors and lysosomal hydrolases. It has been demonstrated that the β-amyloid protein is capable of stimulating the synthesis and secretion of IL-1, IL-6 and IL-8 by microglial cells and thus of activating the cytotoxic mechanisms of acute inflammation (Sabbagh M. N., Galasko D., Thal J. L. (1997) Alzheimer's Disease Review 3, 1-19).

The diseases characterised by deposits of amyloid aggregates include, in addition to Alzheimer's disease, Down's syndrome, hereditary cerebral hemorrhage associated with “Dutch-type” amyloidosis, amyloidosis associated with chronic inflammation, amyloidosis associated with multiple myeloma and other dyscrasias of the haematic B lymphoid cells, amyloidosis associated with type II diabetes, amyloidosis associated with prion diseases such as Creutzfeldt-Jakob disease, Gerstmann-Straussler syndrome, Kuru and the sheep disease scrapie.

In general, however, the damage caused by βA can be summarised as:

1. abnormalities of amyloidogenesis;
2. increase in vulnerability of neurons to exocytoxicity;
3. increase in vulnerability of neurons to hypoglycemic damage;
4. abnormalities of calcium homeostasis;
5. increase in oxidative damage;
6. activation of inflammatory mechanisms;
7. activation of the microglia;
8. induction of lysosomal proteases;
9. abnormalities of tau protein phosphorylation;
10. induction of apoptosis;
11. damage to membranes.

From a strictly theoretical point of view, the reduction of βA-induced damage can be tackled via different therapeutic approaches:
1. reducing the production of βA using secretase inhibitors to alter APP metabolism (increasing α or reducing β and γ secretases);
2. preventing or blocking βA aggregation;
3. increasing βA clearance;
4. blocking the neurotoxic effects of βA by restoring calcium homeostasis;
5. preventing the toxicity produced by free radicals;
6. preventing exocytoxicity;
7. reducing the damage caused by the inflammatory response;
8. correcting the altered copper-zinc equilibrium;
9. inhibiting neuronal apoptosis;

(Sabbagh M. N., Galasko D., Thal J. L. (1997) Alzheimer's Disease Review 3, 1-19).

To date there is no specific therapy capable of preventing, slowing or arresting the amyloidogenic process underlying Alzheimer's disease.

In fact, the therapies currently used for the treatment of this disease are exclusively symptomatic and, though acting on different aspects, interfere fundamentally only with the neurotransmitter mechanisms regulating learning and memory. Among the molecules most commonly used figure the reversible acetylcholinesterase inhibitors such as tacrine, donezepil and rivastigmine.

At the present time, moreover, the only diagnostic instruments available for the diagnosis of Alzheimer's disease are behavioural examinations and clinical scores, while radiographic or scintigraphic procedures are still unable to distinguish with precision between Alzheimer-type forms of degeneration and other degenerative phenomena, the precise reason for this being the lack of suitable tracings.

The difficulties encountered in the management of Alzheimer's disease, its severity and the difficulty in diagnosing it make it desirable not only to identify new drugs capable of curing the disease or of slowing down its course but also to discover compounds to be used in radiographic or scintigraphic procedures for its diagnosis.

It is therefore surprising that pamoic acid, or one of its derivatives, or one of its analogues, or one of the pharmaceutically acceptable salts thereof, or derivatives of said acid described and known in the literature have proved to be potentially effective drugs in the treatment and prevention of Alzheimer's disease and of diseases characterised by deposits of amyloid aggregates.

In the context of this discovery, new derivatives of pamoic acid have been found, described here below, which are potentially effective in the treatment of the above-mentioned diseases and which have proved to be useful agents for the preparation of a medicament for the treatment of diseases characterised by deposits of amyloid aggregates.

In fact, those derivatives of pamoic acid with general formula (I)

| | |
|---|---|
| | (I) |
| 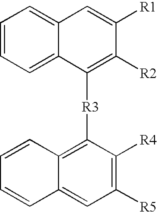 | |

in which:

| | |
|---|---|
| 1 | R1 = R5 = —COOCH₂C₆H₅ |
| | R2 = R4 = —OH |
| | R3 = —CH₂— |
| 2 | R1 = R5 = —COOCH(CH₃)₂ |
| | R2 = R4 = —OH |
| | R3 = —CH₂— |
| 3 | R1 = R5 = —COOC₂H₅ |
| | R2 = R4 = —OH |
| | R3 = —CH₂— |
| 4 | R1 = R5 = —COOC₆H₁₁ |
| | R2 = R4 = —OH |
| | R3 = —CH₂— |
| 5 | R1 = R5 = —COOCH₃ |
| | R2 = R4 = —OH |
| | R3 = —CH₂— |
| 6 | R1 = R5 = —COOC(CH₃)₃ |
| | R2 = R4 = —OH |
| | R3 = —CH₂— |

are described in patent N^{o} ES 432416, and for these
compounds no use is described or claimed;

| | |
|---|---|
| 7 | R1 = R5 = —CONHC₆H₅ |
| | R2 = R4 = —OH |
| | R3 = —CH₂— |

is described in patent N^{o} JP 7138347, as a useful agent for the
preparation of nylon fibres;

| | |
|---|---|
| 8 | R1 = R5 = —CONH—CH(CH(CH₃)₂)—COOH |
| | R2 = R4 = —OH |
| | R3 = —CH₂— |

is described in Reetz, Manfred T. et al; Chem. Commun,
(Cambridge) (1998), (19), 2075-2076 as an inhibitor of HIV-1
protease;

| | |
|---|---|
| 9 | R1 = R5 = —COOH |
| | R2 = R4 = —OCOCH₃ |
| | R3 = —CH₂— |

is described in Poupelin, Jean Pierre; Eur. J. Med. Chem.-
Chim. Ther. (1978), 13(4), 381-5, as an agent with anti-inflammatory
activity;

| | |
|---|---|
| 10 | R1 = R5 = —COOH |
| | R2 = R4 = —OCOCH₂CH₃ |
| | R3 = —CH₂— |

is described in patent N^{o} DE 1945254, which states that the
salts of this compound with streptomycin makes its effect longer-
lasting as an agent for the treatment of tuberculosis;

| | |
|---|---|
| 11 | R1 = R5 = —H |
| | R2 = R4 = —OCOC₆H₅ |
| | R3 = —CH₂— |
| 12 | R1 = R5 = —H |
| | 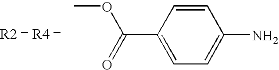 |
| | R3 = —CH₂— |

are described in in Dorogov, M.V.; Khim. Khim. Tekhnol.
(1996), 39 (4-5), 170-172; no use is indicated for them;

| | |
|---|---|
| 13 | R1 = R5 = —H |
| | R2 = R4 = —OCOCH═CH₂ |
| | R3 = —CH₂— |

is described in Kielkiewicz, Jedrzej, et al.; Polimery (Warsaw)
(1984), 29 (6), 216-19; no use is indicated for it;

| | |
|---|---|
| 14 | R1 = R5 = —H |
| | R2 = R4 = —OH |
| | R3 = —CH₂— |

this compound is 1,1′-methylen-di(2-naphtol), which is
described in U.S. Pat. No. 4,147,806 as anti-inflammatory and analgesic
medicament.

| | |
|---|---|
| 15 | R1 = R5 = —COOH |
| | R2 = R4 = —OH |
| | R3 = —CH₂— |

this compound is pamoic acid; it is described as an agent useful as a counter-ion in drugs used as antihelminthic agents (Pyrantel pamoate) or in the treatment of cancer (Octreotide pamoate).

The object of the invention described herein is therefore the use of pamoic acid, or one of its derivatives, or one of its analogues, or one of the pharmaceutically acceptable salts of these, with general formula (I)

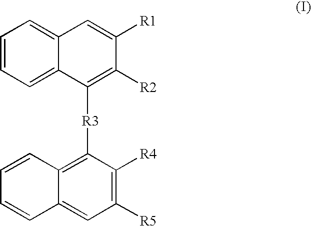

in which:

R1 and R5, which may be the same or different, are COOR6, CONHR6, SO₂R6, SO₂NHR6, SO₃R6, OR6, COR6, NHR6, R6;

in which R6 is H or a straight or branched, saturated or unsaturated alkyl chain, with from 1 to 5 carbon atoms, or phenyl, substituted by R7;

in which: R7 is OH, COOH, SO₃H, NR8R9,

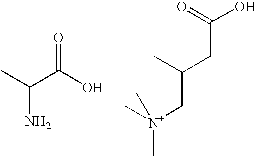

in which:

R8 and R9, which may be the same or different, are H, alkyl with 1 to 5 carbon atoms;

R2 and R4, which may be the same or different, are H, OH, NHR6, OCO—R10-NR8R9,

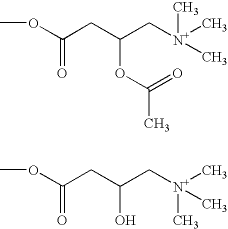

in which R10 is a straight or branched, saturated or unsaturated alkyl chain with from 1 to 5 carbon atoms;

R3 is —[CH₂]n-, —CH₂—O—, —CH(R11)-, in which n is an integer from 1 to 4,

R11 is a straight or branched alkyl with from 1 to 5 carbon atoms, substituted by an amino group, alkylamino C₁-C₅, dialkylamino C₁-C₅, OH, alkyloxy C₁-C₅;

for the preparation of a medicament for the treatment of diseases characterised by deposits of amyloid aggregates.

Among the formula (I) compounds the one preferred is pamoic acid, and particularly sodium pamoate.

A further object of the invention described herein is the use of the above-mentioned formula (I) compounds for the preparation of a diagnostic kit for the diagnosis of diseases characterised by deposits of amyloid aggregates.

In fact, the compounds according to the invention described herein may contain in their molecular structure atoms of elements commonly used in diagnostic imaging procedures. For example, radioactive isotopes of carbon, hydrogen, nitrogen, oxygen, iodine and indium can be introduced into their molecular structure. More precisely, the formula (I) compound can have at least one of the elements, carbon, hydrogen, nitrogen, oxygen of its own molecular structure substituted by a corresponding radioactive isotope; or it will carry at least one atom of radioactive iodine; or it is in the form of a complex with radioactive indium.

Such isotopes are useful for techniques such as PET (Positron Emission Tomography), SPECT (Single Photon Emission Computerized Tomography), and planar scintigraphy. Alternatively, the compounds according to the invention, whether or not they contain radioactive isotopes or atoms of elements useful as radio-opaque substances (e.g. iodine), can be used as complexing agents for elements commonly used in diagnostic imaging techniques, such as, for example, gadolinium (NMR) and technetium (scintigraphy techniques).

On the basis of this diagnostic application, the compounds according to the invention are also useful for the prevention of the diseases indicated above.

A further object of the invention described herein are new compounds with general formula (I)

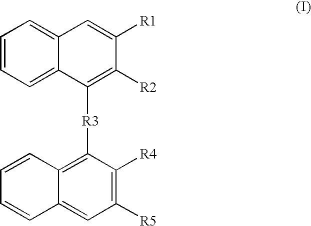

in which:

R1 and R5, which may be the same or different, are COOR6, CONHR6, SO₂R6, SO₂NHR6, SO₃R6, OR6, COR6, NHR6, R6;

in which:

R6 is H or a straight or branched, saturated or unsaturated alkyl chain with from 1 to 5 carbon atoms, or phenyl, substituted by R7;

in which:

R7 is OH, COOH, SO₃H, NR8R9,

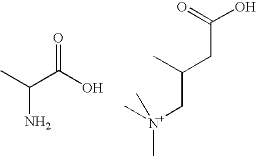

in which:

R8 and R9, which may be the same or different, are H, alkyl with from 1 to 5 carbon atoms;

R2 and R4, which may be the same or different, are H, OH, NHR6, OCO—R10-NR8R9,

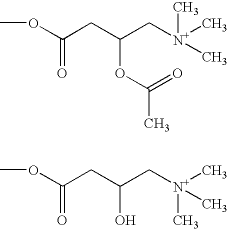

in which:

R10 is a straight or branched, saturated or unsaturated alkyl chain with from 1 to 5 carbon atoms;

R3 is —[CH₂]ₙ—, —CH₂—O—, —CH(R11)-, in which n is an integer from 1 to 4,

R11 is a straight or branched alkyl with from 1 to 5 carbon atoms, substituted by an amino group, alkylamino C1-C5, dialkylamino C1-C5, OH, alkyloxy C1-C5;

with the proviso that the substituents R1, R2, R3, R4 and R5 are not:

| | |
|---|---|
| 1 | R1 = R5 = —COOCH₂C₆H₅ |
| | R2 = R4 = —OH |
| | R3 = —CH₂— |
| 2 | R1 = R5 = —COOCH(CH₃)₂ |
| | R2 = R4 = —OH |
| | R3 = —CH₂— |
| 3 | R1 = R5 = —COOC₂H₅ |
| | R2 = R4 = —OH |
| | R3 = —CH₂— |
| 4 | R1 = R5 = —COOC₆H₁₁ |
| | R2 = R4 = —OH |
| | R3 = —CH₂— |
| 5 | R1 = R5 = —COOCH₃ |
| | R2 = R4 = —OH |
| | R3 = —CH₂— |
| 6 | R1 = R5 = —COOC(CH₃)₃ |
| | R2 = R4 = —OH |
| | R3 = —CH₂— |
| 7 | R1 = R5 = —CONHC₆H₅ |
| | R2 = R4 = —OH |
| | R3 = —CH₂— |
| 11 | R1 = R5 = —H |
| | R2 = R4 = —OCOC₆H₅ |
| | R3 = —CH₂— |
| 12 | R1 = R5 = —H |
| | 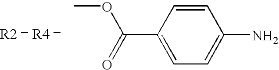 |
| | R3 = —CH₂— |
| 13 | R1 = R5 = —H |
| | R2 = R4 = —OCOCH═CH₂ |
| | R3 = —CH₂—; |
| 14 | R1 = R5 = —H |
| | R2 = R4 = —OH |
| | R3 = —CH₂— |
| 15 | R1 = R5 = —COOH |
| | R2 = R4 = —OH |
| | R3 = —CH₂— |

A further object of the invention described herein is a process for the preparation of compounds with general formula (I)

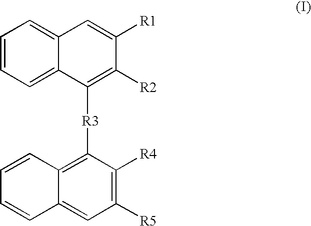

in which:

R1 and R5 are —COOR6,

in which R2, R3, R4 and R5 have the meanings defined above,

characterised in that a general formula (I) compound in which R6 is H, is treated with a halogenating agent, such as SOCl₂ or PCl₅, to yield the corresponding acyl chloride, then reacted at a temperature ranging from 25 to 60° C. for time periods ranging from 2 to 24 hours, under stirring with an R6-OH alcohol in a molar ratio of 1 to 6, or in an inert anhydrous solvent, such as, for example, dimethylformamide, with the stoichiometric amount of R6-OH.

A further object of the invention described herein is a process for the preparation of formula (I) compounds

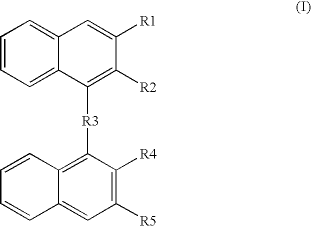

in which R1 and R5 are CONHR6;

in which R2, R3, R4 and R6 have the meanings defined above,

characterised in that a compound with general formula (I), in which R6 is H, is treated with a halogenating agent such as SOCl₂ or PCl₅, to yield the corresponding acyl chloride, or with a coupling agent such as DCC, EEDQ, then reacted at a temperature ranging from 25 to 60° C., for times periods ranging from 2 to 24 hours, under stirring, with an R6-NH₂ amine in a molar ratio of 6 to 1, or in an inert anhydrous solvent with the stoichiometric amount of R6-NH₂.

A further object of the invention described herein is a process for the preparation of formula (I) compounds

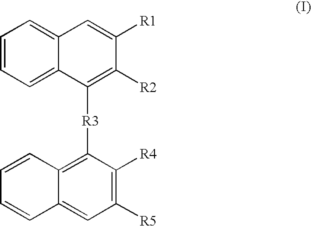

in which R2 and R4 are OH;

in which R1 and R5 are SO₃R6, SO₂NHR6;

R3 is —CH(R11)-,

in which R11 has the meaning indicated above;

characterised in that said process is carried out according to reaction scheme 1 below, where a formula “a” compound is reacted with an R11-CHO aldehyde in glacial acetic acid at a temperature ranging from 90° C. to 150° C. to yield compounds with general formula “b”. Subsequently, a general formula “b” compound is treated with a halogenating agent, such as SOCl2 or PCl5, to yield the corresponding sulphonyl chloride, then reacted with an R6-OH alcohol to yield compounds with general formula “d” or with an R6-NH₂ amine to yield compounds with general formula “e”.

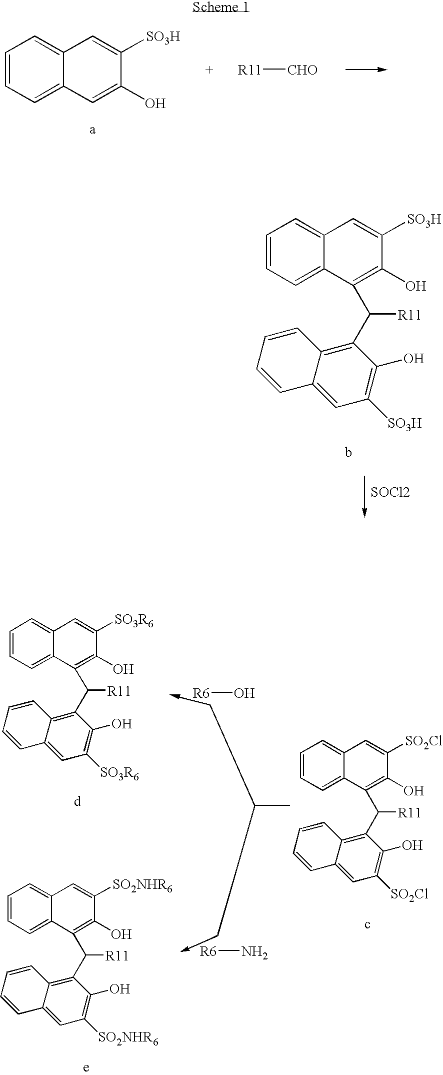

A further object of the invention described herein is a process for the preparation of formula (I) compounds

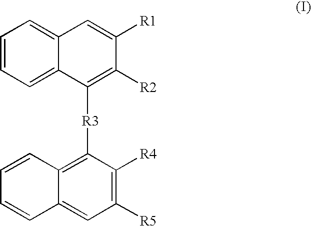

in which:

R1, R2, R4 and R5 are OR6 and/or NHR6; R3 is —CH(R11)-,

in which R6 and R11 have the meanings indicated above; characterised in that said process is carried out according to reaction scheme 2 below, where a formula A compound is reacted with R11-CHO aldehyde in an acid milieu, for example in acetic acid, to yield a mixture of compounds corresponding to the structures B, C and D which are separated and purified by chromatography. These compounds are reacted with an alkyl halide R6-X in the presence of a base and then deprotected in an acid or basic milieu to yield the corresponding naphthyl ethers E, F and G. After treatment of the latter with NaNO₂ in sulphuric acid, compounds H, I and L are obtained.

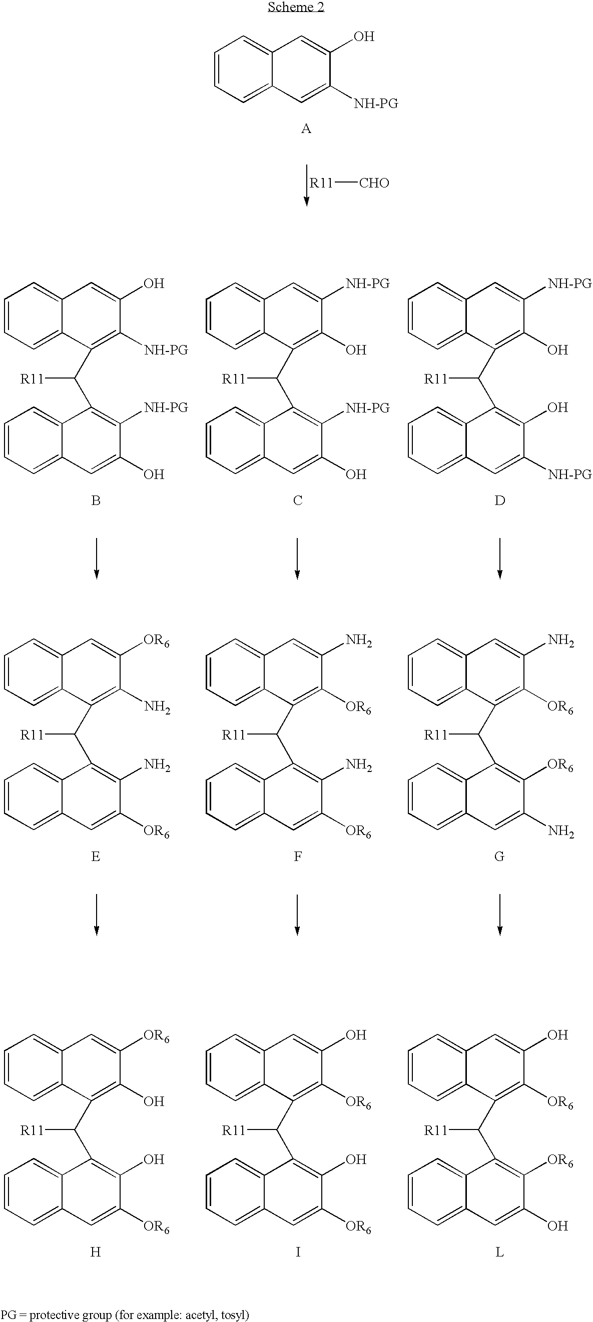

A further object of the invention described herein is a pharmaceutical composition containing as active ingredient a compound with general formula (I)

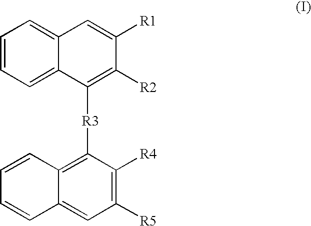

in which R1, R2, R3, R4 and R5 have the meanings indicated above,

with the proviso that R1, R2, R3, R4 and R5 are not:

| | |
|---|---|
| 1 | R1 = R5 = —COOCH₂C₆H₅ |
| | R2 = R4 = —OH |
| | R3 = —CH₂— |
| 2 | R1 = R5 = —COOCH(CH₃)₂ |
| | R2 = R4 = —OH |
| | R3 = —CH₂— |
| 3 | R1 = R5 = —COOC₂H₅ |
| | R2 = R4 = —OH |
| | R3 = —CH₂— |
| 4 | R1 = R5 = —COOC₆H₁₁ |
| | R2 = R4 = —OH |
| | R3 = —CH₂— |
| 5 | R1 = R5 = —COOCH₃ |
| | R2 = R4 = —OH |
| | R3 = —CH₂— |
| 6 | R1 = R5 = —COOC(CH₃)₃ |
| | R2 = R4 = —OH |
| | R3 = —CH₂— |
| 7 | R1 = R5 = —CONHC₆H₅ |
| | R2 = R4 = —OH |
| | R3 = —CH₂— |
| 11 | R1 = R5 = —H |
| | R2 = R4 = —OCOC₆H₅ |
| | R3 = —CH₂— |
| 12 | R1 = R5 = —H |
| | 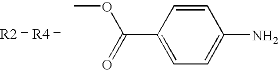 |
| | R3 = —CH₂— |
| 13 | R1 = R5 = —H |
| | R2 = R4 = —OCOCH═CH₂ |
| | R3 = —CH₂—; |
| 14 | R1 = R5 = —H |
| | R2 = R4 = —OH |
| | R3 = —CH₂— |
| 15 | R1 = R5 = —COOH |
| | R2 = R4 = —OH |
| | R3 = —CH₂— |

and a pharmaceutically acceptable excipient and/or diluent.

Given here below are a number of examples which further illustrate the invention.

### EXAMPLE 1

### Preparation of (2R)-2-(acetyloxy)-4-({3-carboxy-1-[(3-carboxy-2-hydroxy-1-naphthyl)methyl]-2-naphthyl}oxy)-N,N,N-trimethyl-4-oxo-1-butanaminium chloride (ST1722)

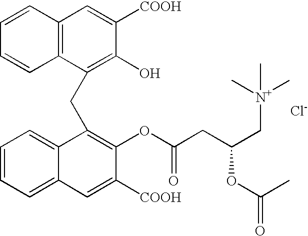

A solution of 2.39 g (0.01 mol) of acetyl L-carnitine chloride, 2 ml of anhydrous CH₂Cl₂, and 1.1 ml (0.015 mol) of thionyl chloride was stirred at ambient temperature for 4 hours. The solvent was removed and the residual solid washed three times with anhydrous CH₂Cl₂. An oil was obtained, namely the acyl chloride of acetyl L-carnitine chloride, which was used as such for the next step.

A suspension of 2.58 g (0.01 mol) of acyl chloride of acetyl L-carnitine chloride, 3.88 g (0.01 mol) of pamoic acid and 10 ml of N-methyl-2-pyrrholidinone was left to stir for one night. After precipitation with ethyl ether, a yellow solid was obtained (7 g). The crude product thus obtained was purified by chromatography on a silica gel column, eluting first with CH₂Cl₂-MeOH 90:10 to collect the unreacted pamoic acid and then with CH₂Cl₂-MeOH 85:15 to collect the product. After removal of the solvent, 1.2 grams of (2R)-2-(acetyloxy)-4-({3-carboxy-1-[(3-carboxy-2-hydroxy-1-naphthyl)methyl]-2-naphthyl}oxy)-N,N,N-trimethyl-4-oxo-1-butanaminium chloride were obtained.

Yield=19.7%, M.P.=decomposes at 185° C., [α]_{D}²⁰=−17.5°,

¹H NMR (DMSO, 300 MHz), δ 7.1-8.5 (m, 10H, H—Ar), 5.50 (m, 1H, —C—CH—C—N), 4.76 (s, 2H, Ar—CH₂—Ar), 3.70 (m, 2H, —CH₂—N), 3.11 (s, 9H, —N—CH₃), 2.85 (m, 2H, —CH₂—COO—), 2.01 (s, 3H, CH₃—COO—).

K.F.=1.4%

C, H, N values calculated for C₃₂H₃₂NO₉Cl and corrected for the amount of water present: C, 63.00; H, 5.29; N, 2.30; found C, 60.45; H, 5.83; N, 2.87.

### EXAMPLE 2

### Preparation of (2R)-2-(acetyloxy)-4-({1-[(2-hydroxy-1-naphthyl)methyl]-2-naphthyl}oxy)-N,N,N-trimethyl-4-oxo-1-butanaminium chloride (ST1745)

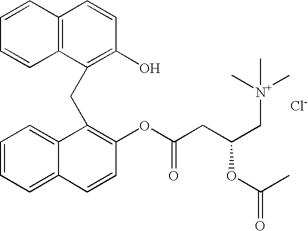

A solution of 2.39 g (0.01 mol) of acetyl L-carnitine chloride, 2 ml of anhydrous CH₂Cl₂, and 1.1 ml (0.015 mol) of thionyl chloride was stirred at ambient temperature for 4 hours. The solvent was removed and the residual solid washed three times with anhydrous CH₂Cl₂. An oil was obtained, namely the acyl chloride of acetyl L-carnitine chloride, which was used as such for the next step.

To a solution of 2.58 g (0.01 mol) of acyl chloride of acetyl L-carnitine chloride in CH₃CN (5 ml) was added 3 g (0.01 mol) of 1,1′-methylene-di(2-naphthol) (ST1859). The mixture was stirred at room temperature overnight. After precipitation with ethyl ether a crude product was obtained. This product was washed with diethyl ether, dried under vacuum, and purified by silica-gel chromatography (9:1 CH₂Cl₂/MeOH mixture). The fractions contained the product, controlled by TLC, were combined. The solvent was removed to give 2 g (0.0038 mol) of (2R)-2-(acetyloxy)-4-({1-[(2-hydroxy-1-naphthyl)methyl]-2-naphthyl}oxy)-N,N,N-trimethyl-4-oxo-1-butanaminium chloride (ST1745). Yield=38%

¹H NMR (DMSO, 300 MHz), δ 10.05 (s, 1H, —OH), 7.15-8.3 (m, 12H, H—Ar), 5.55 (m, 1H, —C—CH—C—N), 4.65 (s, 2H, Ar—CH₂—Ar), 3.6-3.9 (m, 2H, —CH₂—N), 3.10 (s, 9H, —N—CH₃), 2.95 (m, 2H, —CH₂—COO—), 2.00 (s, 3H, CH₃—COO—)

K.F.=4.4%

C, H, N values calculated for C₃₀H₃₂NO₅Cl and corrected for the amount of water present: C, 69.02; H, 6.18; N, 2.68; found C, 68.6; H, 6.3; N, 2.61.

### EXAMPLE 3

### Preparation of 2-({1-[(2-hydroxy-1-naphthyl)methyl]-2-naphthyl}oxy)-2-oxoethanaminium chloride (ST1913)

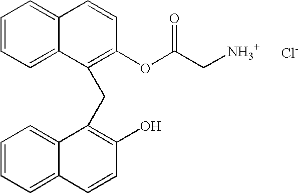

To a solution of 2 g (0.011 mol) of N-(tert-butoxycarbonyl)-glycine (BOC-GLY-OH) in 2 ml of toluene was added 0.62 g (0.011 mol) of KOH and 2 ml of H₂O.

The mixture was undergone to azeotropic distillation (150° C.) in order to eliminate the water. The obtained solution was cooled at 0° C. and 0.85 ml of isobutanol, 11 μl (d=0.92, 0.1 mmol) of N-methyl-morfolin, and 1.68 ml of isobutyl chloroformiate (d=1.044, 0.0128 mol) was added. The reaction mixture was stirred at 0° C. for 2 h.

Subsequently, a solution of 1.65 g of 1,1′-methylen-di(2-naphtol) (ST1859) (0.0055 mol) and 0.62 g of KOH in 15 ml of H₂O was prepared. Such solution was added to reaction mixture and was stirred at room temperature. After 1 h the pH was adjusted to a 3 with HCl 3N and the phases were separated. The organic phase, toluene, was extracted with 20 ml of H₂O adjusted to a pH of 9 with NaOH 3N and washed with H₂O until neutrality. The separated organic phase was dried over Na₂SO₄ and the solvent was removed to give a crude product. After recrystallization from n-exane/ethyl acetate 8:2 0.2 g of product was obtained that were dissolved in 1 ml of trifluoroacetic acid for tert-butoxy-carconyl hydrolysis. After 20 min was obtained the precipitation of a solid which was filtered, and washed with a mixture of n-exane/diethyl ether 8:2. The obtained product was dissolved in methanol and got through a A21/Cl-resin eluating with 100 ml of MeOH to give 60 mg of 2-({1-[(2-hydroxy-1-naphthyl)methyl]-2-naphthyl}oxy)-2-oxoethanaminium chloride (ST1913).

¹H NMR (DMSO, 300 MHz), δ 9.6 (s, 1H, —OH), 8.7 (s, 3H, —NH₃), 7.2-8.4 (m, 12H, H—Ar), 4.7 (s, 2H, Ar—CH₂—Ar), 3.72 (s, 2H, C—CH₂—N).

K.F.=1.2%

C, H, N values calculated for C₂₃H₂₀NO₃Cl and corrected for the amount of water present: C, 70.14; H, 5.12; N, 3.56; found C, 69.1; H, 5.4; N, 3.3.

### EXAMPLE 4

### Preparation of 2-({4-[(3-{[2-(diethylammonio)ethoxy]carbonyl}-2-hydroxy-1-naphthyl)methyl]-3-hydroxy-2-naphthoyl}oxy)-N,N-diethylethanaminium dichloride (ST1800)

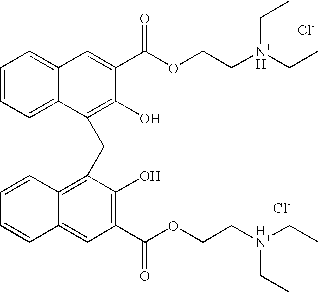

3.88 g (0.01 mol) of pamoic acid (ST1641) was suspended in 4.36 ml of thyonil chloride (0.06 mol) and refluxed at 80° C. for 5 h. At the end, the solvent was removed under vacuum and the residue was washed with diethyl ether. The acylic chloride obtained was suspended in 30 ml of CH₂Cl₂ and 0.7 ml of N,N-diethyl ethanol was added dropwise. The mixture was stirred at room temperature overnight. At the end a white solid was obtained which was filtered and washed with a mixture of n-exane/ethyl acetate 8:2 to give 0.5 g 2-({4-[(3-{[2-(diethylammonio)ethoxy]carbonyl}-2-hydroxy-1-naphthyl)methyl]-3-hydroxy-2-naphthoyl}oxy)-N,N-diethylethanaminium dichloride (ST1800).

¹H NMR (CDCl₃, 300 MHz), δ 10.05 (s, 2H, —OH), 7.1-8.4 (m, 10H, H—Ar), 4.85 (s, 2H, Ar—CH₂—Ar), 4.55 (t, 4H, —O—CH₂—CH₂—N), 3.0 (t, 4H, —O—CH₂—CH₂—N), 2.75 (m, 8H, —N—CH₂—CH₃), 1.0 (t, 12H, —N—CH₂—CH₃).

K.F.=0.8% C, H, N values calculated for C₃₅H₄₄N₂O₆Cl₂ and corrected for the amount of water present: C, 63.72; H, 6.72; N, 4.24; found C, 63.5; H, 5.87; N, 4.6.

### EXAMPLE 5

### Evaluation of Antiaggregant Effects of Sodium Pamoate (ST1641) on β-amyloid₂₅₋₃₅ Peptide

To 250 μl of a solution consisting of sodium pamoate 2 mM and phosphate buffer 200 mM pH 5 were added 250 μl of an aqueous solution of βA₂₅₋₃₅ 2 mM (cat. Bachem n° H-1192.0001). 500 μl of a solution of sodium pamoate 1 mM, βA₂₅₋₃₅ 1 mM and phosphate buffer 100 mM pH 5 were thus obtained.

The same process was carried out for the control sample where sodium pamoate was not present.

After 24 hours at ambient temperature, the sample and control were centrifuged at 12000 rpm for 20 minutes, separating the settled solids from the supernatants. To the settled solids were added 250 μL of water. After 3 hours at ambient temperature the samples were centrifuged again at a 12000 rpm for 20 minutes. After centrifuging, no presence of any solid was observed in the sample, unlike the control. This result demonstrated the complete inhibition of aggregation of βA₂₅₋₃₅ peptide in fibrils by sodium pamoate.

### EXAMPLE 6

### Evaluation of Antiaggregant Effects of Sodium Pamoate (ST1641) on β-amyloid₁₋₄₂ Peptide

The antiaggregant effects of sodium pamoate on βA₁₋₄₂ peptide were evaluated by measuring thioflavin T binding according to the following procedure.

βA₁₋₄₂ peptide (cat. Bachem n° H-1368.0500) at a concentration of 0.22 mM was incubated at 37° C. in Tris buffer 100 mM pH 7.4, alone or in the presence of sodium pamoate, for 5 days. The molar ratios of the peptide to sodium pamoate were generally 1:8, 1:4 and 1:2.

The solution was centrifuged at 13000 rpm for 5 minutes and the supernatant was eliminated. The precipitate was washed with 500 μl of H₂O and centrifuged at 13000 rpm for 5 minutes. In the precipitate, the aggregate in fibril form was detected with 600 μl of thioflavin T (ThT) 2 μM dissolved in glycine-NaOH buffer 50 mM, pH 9.4. After 5 minutes' incubation 500 μl of samples were transferred to a quartz cuvette and the fluorimetric signal was determined at 420 nm excitation and 480 nm emission in a spectrophotofluorimeter. In these conditions the fluorimetric signal is proportional to the amount of amyloid aggregate (Le Vine, Methods in Enzymology, vol. 309 pp 274-284).

Sodium pamoate, in this experiment, proved capable of producing a consistent and dose-dependent reduction in the formation of βA₁₋₄₂ aggregates in the form of fibrils. The effect is significant and the reduction reaches as much as 70% as compared to controls.

The inhibition of fibril formation was also measured as a function of incubation time. On going from 1 to 5 days' incubation, sodium pamoate showed a progressive increase in efficacy in reducing thioflavin T binding.

### EXAMPLE 7

### Evaluation of Antiaggregant Effects of ST Compounds on β-amyloid₁₋₄₂ Peptide

The ST1641, ST1722, ST1859, ST1745, ST1800, ST1913 capability to counteract βA₁₋₄₂ polymerization was evaluated using the Thioflavina “T” binding assay with the following procedure (M. A. Findeis, S. M. Molineaux; Methods in Enzymology 309, 487-488 (1999)): βA₁₋₄₂ peptide (1 mg/ml) was dissolved in H₂O/CH₃CN (1:1), lyophilized, solubilized in DMSO+PBS and incubated at 37° C. for 8 days. The peptide was then sonicated and dissolved in PBS (1:5). 96 well plates were prepared with a solution of βA₁₋₄₂ (40 μl/well) and ST testing compounds (50 μl/well, at concentrations between 0.8 and 100 μM). 50 μl of not aggregated βA₁₋₄₂ was added after 15 minutes to each well and the plates were incubated overnight at 37° C. with agitation. 200 μl of a reaction mixture containing Thioflavina “T” (10 μM) and Na₂HPO₄×2H₂O (50 μM) solution (pH 6.5) was then added to each well. The fluorescence was measured at 450 nm of excitation and 482 nm of emission with a 96 well fluorimetric plate reader within 60 seconds. At this experimental conditions fluorimetric measures were related to the amount of βA₁₋₄₂ polymerized peptide.

Table 1 shows the DE₅₀ values of ST tested compounds.

TABLE 1

| Compound | DE₅₀ (μM) |
|---|---|
| ST1641 | 38.2 |
| ST1745 | 90.3 |
| ST1859 | 5.4 |
| ST1745 | 8.0 |
| ST1800 | >50 |
| ST1913 | 7.8 |

### EXAMPLE 8

### Dissolution of Aggregates of Preformed β-amyloid₁₋₄₂ in Fibril Form by Sodium Pamoate (ST1641)

This experiment was conducted in order to assess the antiaggregant capacity of sodium pamoate on previously aggregated βA₁₋₄₂ peptide, according to the following procedure.

βA₁₋₄₂ peptide was left to aggregate for 48 hours at 37° C. in the conditions described in Example 6. Sodium pamoate was added (peptide:pamoate ratio 1:8).

In these conditions, sodium pamoate proved extremely active in reducing thioflavin T binding.

Incubation with sodium pamoate led to a 70% reduction in fluorescence as compared to controls not incubated with sodium pamoate.

This result demonstrates that sodium pamoate was capable of exerting an antiaggregant effect a posteriori on the fibrillar structure of βA₁₋₄₂.

### EXAMPLE 9

### Reduction of Resistance of β-amyloid₁₋₄₂ Peptide to Trypsin Digestion Induced by Sodium Pamoate (ST1641)

βA₁₋₄₂ peptide was dissolved with 15 μl of NaOH 0.1 M. The solution was brought to pH 7.4 with 15 μl of TRIS buffer 100 mM to which were added 30 μl of buffer alone or 30 μl of buffer solution containing sodium pamoate. The final concentration of βA₁₋₄₂ peptide was 0.22 mM, and that of sodium pamoate ranged from 0.055 to 1.76 mM, thus with a βA₁₋₄₂ peptide:sodium pamoate ratio ranging from 4:1 to 1:8.

The samples thus prepared were incubated at 37° C. for 5 days; in these conditions βA₁₋₄₂ peptide formed aggregates in the form of fibrils modifying its structure from random-coil to β-sheet (Zagorski M. G. et al. 1999 “Methodological and Chemical Factors Affecting Amyloid β Peptide Amyloidogenicity” Methods in Enzymology 309:189-204). After 5 days' incubation, 24 μg of trypsin (Merck) were added to each sample, stirred and centrifuged for 1 minute at 13000 rpm; the samples were then left to incubate at 37° C. for 1 hour.

When this period had elapsed, the mixture was centrifuged for 5 minutes at 13000 rpm, eliminating 50 μl of supernatant, and the precipitate was dissolved with 40 μl of HCOOH and 10 μl of H₂O containing 0.1% of trifluoroacetic acid (TFA).

At this point the sample was ready for quantitative HPLC analysis. The HPLC profile of the sample incubated with sodium pamoate was compared with that obtained with peptide alone, thereby quantifying the βA₁₋₄₂ peptide.

Trypsin, in the conditions described above, was capable of hydrolysing from 30 to 50% of the βA₁₋₄₂ peptide. The trypsin hydrolysis of βA₁₋₄₂ was increased by sodium pamoate by more than 50% at the highest dose (peptide:pamoate ratio 1:8) and by more than 40% at the lowest dose (1:4).

### EXAMPLE 10

### Sodium Pamoate (ST1641) Inhibition of Neurotoxicity Induced by β-amyloid₂₅₋₃₅

To verify the potential neuroprotective activity of sodium pamoate, primary cortical neuronal cultures obtained by microdissection of rat foetal brain at day 16-18 of gestation were used. The cerebral tissue was cultivated in the presence of foetal calf serum and the glial proliferation was inhibited by adding to the incubation medium the antimitotic agent cytosine arabinoside on days 3 and 5 (Andreoni et al. 1997 Exp. Neurology 148:281-287). The cell cultures were exposed to βA₂₅₋₃₅ peptide for 5-7 days in the presence or absence of sodium pamoate. The neuroprotective action was evaluated in conditions of neurotoxicity induced by kainic acid to verify the specificity of action of sodium pamoate and its effective antiaggregant activity against the neurotoxic agent. The ability of sodium pamoate to protect the cells against degeneration was also evaluated in neuronal cells cultured in the absence of foetal calf serum in the culture medium. In this case, 24 hours after seeding, the medium was replaced with one without serum containing glutamine, insulin, transferrin, putrescin, progesterone, sodium selenite and Hepes.

### Experimental Procedure

Primary cultures of neurons of the cerebellar cortex were taken from the rat foetal brain on days 16-18 of gestation and cultured in foetal calf serum. On incubation days 3 and 5, glial proliferation was inhibited using cytosine arabinoside as an antimitotic agent.

The cultures were exposed to βA₂₅₋₃₅ peptide at concentrations of 25 and 50 μM from the day following seeding for 5 to 7 days.

βA₂₅₋₃₅ peptide was added to the cultures together with sodium pamoate which had equimolar concentrations or concentrations lower than those of the peptide itself.

The protection against neurotoxicity was evaluated using the colorimetric method and densitometric analysis with an image analyser.

The results obtained show that sodium pamoate was capable of affording complete protection against the toxicity induced by βA₂₅₋₃₅. The results obtained are given in the Table 2.

TABLE 2

| | | | Sodium pamoate |
|---|---|---|---|
| | βA₂₅₋₃₅ | Sodium pamoate | 25 μM + |
| Control | 50 μM | 25 μM | βA₂₅₋₃₅ 50 μM |
| % S | % S | % S | % S |
| 100 | 16 | 88 | 100 |

% S: percentage survival

### EXAMPLE 11

### Sodium Pamoate (ST1641) Reduction of Apoptosis of Cerebellar Granules Induced by K⁺ Deprivation

Granules isolated from cerebellum of 8-day-old rats are differentiated biochemically and morphologically in approximately one week, becoming morphologically mature and with a glutamatergic interneuron phenotype (Gallo et al. 1982 PNAS 79:7919-7923). On depriving the culture medium of serum and reducing the extracellular concentration of potassium ions (25 mM) to the extent of bringing it down to a non-depolarising condition (5 mM), cell death by apoptosis is obtained in approximately 24 hours.

Programmed neuronal death is a phenomenon observed not only in numerous physiological processes but also in many neurodegenerative diseases such as AD, Parkinson's disease, Huntington's chorea and amyotrophic lateral sclerosis. In the case of AD, the existence of a close relationship is detected between apoptosis and the presence of βA mutation of the presenile 2 (PS2) gene which regulates the production of amyloid itself. In fact, in cases of AD in which a PS2 mutation is present, a classic increase in cerebral and plasma βA₁₋₄₂ is also detectable (Scheuner D., Eckman C., Jensen M., Song X., Citron M., Suzuki N., Bird T. D., Hardy J., Hutton M., Kukull W., Laeson E., Levy-Lahad E., Viitanen M., Peskind E., Selkoe D., Yunkin S. (1996) Nat. Med. 2, 864-870.); moreover, the mutated form of the PS2 gene, expressed in PC12, causes apoptosis (Wolozin B., Iwasaki K., D'Adamio L. (1996) Science 274, 1710-1713).

This experimental model made it possible to obtain a “self-fuelling” βA production system where neuronal apoptosis of the cerebellar granules brought about changes in the processing of the amyloid precursor APP, of such a nature as to favour the course of amyloidogenic metabolism. The increase in βA levels, in turn, favours programmed cell death. In this experimental setting, the potential efficacy of the study substances was measured in terms of cell survival at given times (24, 48 and 72 hours) after the reduction of KCl in the medium.

### Experimental Procedure

In primary cultures of cerebellar granules of 8-day-old rats, maintained in a culture medium containing KCl 25 mM, the cells were labelled with ³⁵S-methionine after 6 days in culture.

Apoptosis was induced by deprivation of the serum and reduction of the KCl concentration from 25 mM to 5 mM.

This situation represented the neuronal differentiation condition in vitro or resection of the dendritic and axonal branches entering and exiting the nerve tissue cells.

As a result of the apoptosis there was an overproduction of βA.

The cultures were incubated with sodium pamoate at concentrations ranging from 1 μM to 100 μM.

The protection against toxicity was assessed in terms of cell viability at 24, 48 and 72 hours.

### Results

The results obtained in this experiment showed that sodium pamoate, at a concentration of 10 μM, has a protective effect (89% protection) against the damage induced by amyloid forming during the apoptotic process.

### EXAMPLE 12

### ST1859 Capability to Cross “In Vivo” the Blood Brain Barrier

Post-mortem examination of AD brain sections reveals the presence of abundant extracellular senile plaques composed of fibrillar amyloid aggregates. The relationship between the presence of beta amyloid peptide and the severity of the illness suggests that the inhibition of peptide fibril formation may be a potential tool for the therapy of this illness. ST1859 inhibited “in vitro” the beta amyloid aggregation and to test its permeability through the intact blood-brain barrier, ST1859, labelled with ¹⁴C(S. A. 50 μCi/mM), was injected i.v. into normal rats at the dose of 18 μCi/rat. The brain and blood were up-taken 30′ after the injection, the blood was then centrifuged (3000 RPM×15 min) and serum obtained was diluted 1:20 with water while brain tissue was homogenized 1:20 w/v in water. To each sample was then added 4 ml of scintillation liquid for aqueous samples The amount of radioactivity was counted with an automatic β-counter (Packard 4600). Data reported in DPM (table 1) were normalized vs. weight or volume of each sample. Results obtained in this experiment showed that ST1859 is able to cross the blood brain barrier with a rate serum/brain <1 (table 3).

TABLE 3

| | Serum | Brain | |
|---|---|---|---|
| | (DPM/ml) | (DPM/g) | Serum/Brain |
| Mean | 29.776 | 35.643 | 0.833 |
| S.E. | ±1253 | ±1349 | ±0.042 |

1. A compound of the general formula (I) 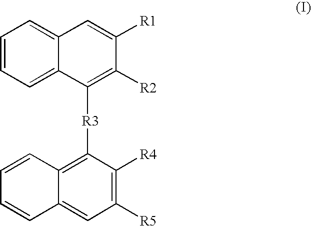
in which:
R1 and R5, which may be the same or different, are COOR6, CONHR6, SO₂R6, SO₂NHR6, SO₃R6, OR6, COR6, NHR6, R6;
in which R6 is H or a straight or branched, saturated or unsaturated alkyl chain, with from 1 to 5 carbon atoms, or phenyl, substituted by R7;
in which:
R7 is OH, COOH, SO₃H, NR8R9, 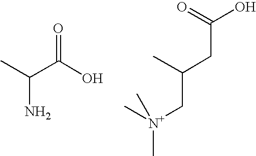
in which:
R8 and R9, which may be the same or different, are H, alkyl with 1 to 5 carbon atoms;
R2 and R4, which may be the same or different, are H, OH, NHR6, OCO—R10-NR8R9, 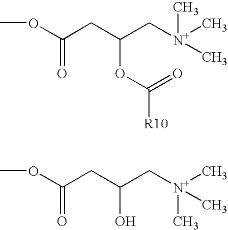
in which R10 is a straight or branched, saturated or unsaturated alkyl chain with from 1 to 5 carbon atoms;
R3 is —[CH₂]n-, —CH₂—O—, —CH(R11)-,
in which n is an integer from 1 to 4, R11 is a straight or branched alkyl with from 1 to 5 carbon atoms, substituted by an amino group, alkylamino C₁-C₅, dialkylamino C₁-C₅, OH, alkyloxy C₁-C₅; and its pharmaceutically acceptable salts;
with the proviso that the substituents R1, R2, R3, R4 and R5 are not:

| | |
|---|---|
| 1 | R1 = R5 = —COOCH₂C₆H₅ |
| | R2 = R4 = —OH |
| | R3 = —CH₂— |
| 2 | R1 = R5 = COOCH(CH₃)₂ |
| | R2 = R4 = —OH |
| | R3 = —CH₂— |
| 3 | R1 = R5 = —COOC₂H₅ |
| | R2 = R4 = —OH |
| | R3 = —CH₂— |
| 4 | R1 = R5 = —COOC₆H₁₁ |
| | R2 = R4 = —OH |
| | R3 = —CH₂— |
| 5 | R1 = R5 = COOCH₃ |
| | R2 = R4 = —OH |
| | R3 = —CH₂— |
| 6 | R1 = R5 = COOC(CH₃)₃ |
| | R2 = R4 = —OH |
| | R3 = —CH₂— |
| 7 | R1 = R5 = —CONHC₆H₅ |
| | R2 = R4 = —OH |
| | R3 = —CH₂— |
| 11 | R1 = R5 = —H |
| | R2 = R4 = —OCOC₆H₅ |
| | R3 = —CH₂— |
| 12 | R1 = R5 = —H |
| | 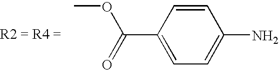 |
| | R3 = —CH₂— |
| 13 | R1 = R5 = —H |
| | R2 = R4 = OCOCH═CH₂ |
| | R3 = —CH₂—; |
| 14 | R1 = R5 = —H |
| | R2 = R4 = —OH |
| | R3 = —CH₂— |
| 15 | R1 = R5 = —COOH |
| | R2 = R4 = —OH |
| | R3 = —CH₂— |
| 16 | R1 = R5 = —COOH |
| | R2 = R4 = —OH |
| | R3 = —CH₂— |
| 17 | R1 = R5 = —COOH |
| | R2 = R4 = —OH |
| | R3 = —CH₂—CH₂— |
| 18 | R1 = R5 = SO₃H |
| | R3 = —CH₂—. |

2.-6. (canceled)

7. A process for the preparation of a compound of the general formula (I) 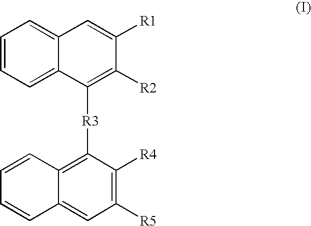
R1 and R5 are —COOR6,
in which R2, R3, R4 and R5 have the meanings defined in claim 1,
wherein the general formula (I) compounds in which R6 is H, is treated with a halogenating agent to yield the corresponding acyl chloride, which is then reacted under stirring with an R6-OH alcohol in a molar ratio of 1 to 6, or in an inert anhydrous solvent with the stoichiometric amount of R6-OH.

8.-25. (canceled)

26. Process for the preparation of a compound of formula (I) 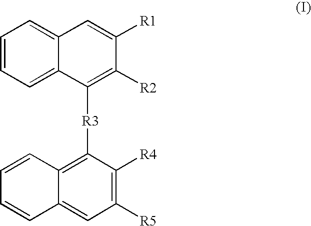
wherein R1, R2, R4 and R5 are OR6 and/or NHR6 and R3 is —CH(R11)-, R6 and R11 have the meanings defined in claim 1;
wherein according to the reaction scheme 2 below, comprising reacting formula A compound with R11-CHO aldehyde in an acid milieu to yield a mixture of compounds corresponding to the structures B, C, and D which are then separated, and purified; then reacting these compounds with an R6-X alkyl halide, in which X is fluorine, chlorine, bromine or iodine, in the presence of a base and then deprotecting them in an acid milieu to yield the corresponding naphthyl ethers E, G, F, then treating these compounds with NaNO2 in sulphuric acid, thereby to obtain compounds H, I and L;

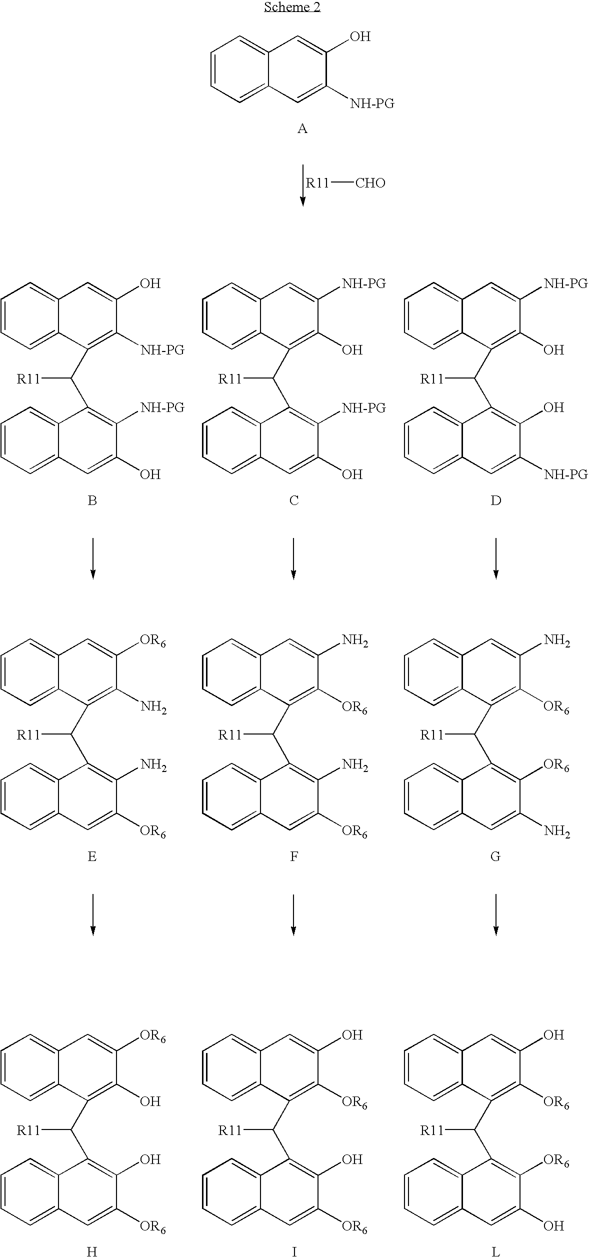

27. A pharmaceutical composition containing as its active ingredient a compound of the formula (I) 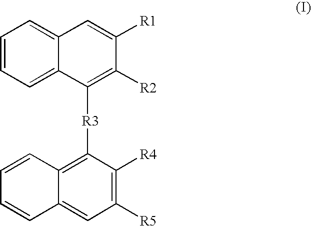
in which:
R1 and R5, which are different, are COOR6, CONHR6, SO₂R6, SO₂NHR6, SO₃R6, OR6, COR6, NHR6, R6;
R1 and R5, which are the same, are COOR6, CONHR6, SO₂R6, SO₂NHR6, OR6, COR6, NHR6, R6;
in which R6 is H or a straight or branched, saturated or unsaturated alkyl chain, with from 1 to 5 carbon atoms, or phenyl, substituted by R7;
in which:
R7 is OH, COOH, SO₃H, NR8R9, 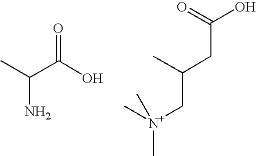
in which:
R8 and R9, which may be the same or different, are H, alkyl with 1 to 5 carbon atoms;
R2 and R4, which may be the same or different, are H, OH, NHR6, OCO—R10-NR8R9, 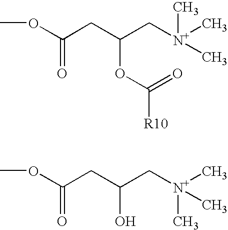
in which R10 is a straight or branched, saturated or unsaturated alkyl chain with from 1 to 5 carbon atoms;
R2 and R4 are OH when R5 is COOR6, CONHR6, SO₂NHR6, SO₃R6, NHR6, R6;
R3 is [CH2]n—, —CH₂—O—, —CH(R11)-,
in which n is an integer from 1, 3 or 4, R11 is a straight or branched alkyl with from 1 to 5 carbon atoms, substituted by an amino group, alkylamino C₁-C₅, dialkylamino C₁-C₅, OH, alkyloxy C₁-C₅; and its pharmaceutically acceptable salts;
with the proviso that the substituents R1, R2, R3, R4 and R5 are not:

| | |
|---|---|
| 1 | R1 = R5 = —COOCH₂C₆H₅ |
| | R2 = R4 = —OH |
| | R3 = —CH₂— |
| 2 | R1 = R5 = COOCH(CH₃)₂ |
| | R2 = R4 = —OH |
| | R3 = —CH₂— |
| 3 | R1 = R5 = —COOC₂H₅ |
| | R2 = R4 = —OH |
| | R3 = —CH₂— |
| 4 | R1 = R5 = —COOC₆H₁₁ |
| | R2 = R4 = —OH |
| | R3 = —CH₂— |
| 5 | R1 = R5 = COOCH₃ |
| | R2 = R4 = —OH |
| | R3 = —CH₂— |
| 6 | R1 = R5 = COOC(CH₃)₃ |
| | R2 = R4 = —OH |
| | R3 = —CH₂— |
| 7 | R1 = R5 = —CONHC₆H₅ |
| | R2 = R4 = —OH |
| | R3 = —CH₂— |
| 11 | R1 = R5 = —H |
| | R2 = R4 = —OCOC₆H₅ |
| | R3 = —CH₂— |
| 12 | R1 = R5 = —H |
| | 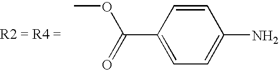 |
| | R3 = —CH₂— |
| 13 | R1 = R5 = —H |
| | R2 = R4 = OCOCH═CH₂ |
| | R3 = —CH₂—; |
| 14 | R1 = R5 = —H |
| | R2 = R4 = —OH |
| | R3 = —CH₂— |
| 15 | R1 = R5 = —COOH |
| | R2 = R4 = —OH |
| | R3 = —CH₂— |

and at least one pharmaceutically acceptable excipient and/or diluent.

28. A method for inhibiting the aggregation of beta-amyloid peptide comprising administering to a subject an effective amount of a compound of the formula (I) 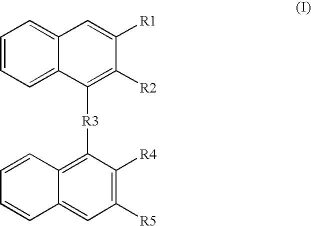
in which:
R1 and R5, which are different, are COOR6, CONHR6, SO₂R6, SO₂NHR6, SO₃R6, OR6, COR6, NHR6, R6;
R1 and R5, which are the same, are COOR6, CONHR6, SO₂R6, SO₂NHR6, OR6, COR6, NHR6, R6;
in which R6 is H or a straight or branched, saturated or unsaturated alkyl chain, with from 1 to 5 carbon atoms, or phenyl, substituted by R7;
in which:
R7 is OH, COOH, SO₃H, NR8R9, 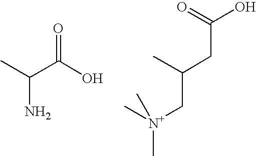
in which:
R8 and R9, which may be the same or different, are H, alkyl with 1 to 5 carbon atoms;
R2 and R4, which may be the same or different, are H, OH, NHR6, OCO—R10-NR8R9, 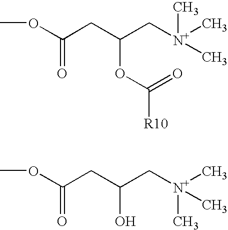
in which R10 is a straight or branched, saturated or unsaturated alkyl chain with from 1 to 5 carbon atoms;
R2 and R4 are OH when R5 is COOR6, CONHR6, SO₂NHR6, SO₃R6, NHR6, R6;
R3 is —[CH₂]n-, —CH₂—O—, —CH(R11)-,
in which n is an integer from 1, 3 or 4, R11 is a straight or branched alkyl with from 1 to 5 carbon atoms, substituted by an amino group, alkylamino C₁-C₅, dialkylamino C₁-C₅, OH, alkyloxy C₁-C₅; and its pharmaceutically acceptable salts
with the proviso that the substituents R1, R2, R3, R4 and R5 are not:

| | |
|---|---|
| 1 | R1 = R5 = —COOCH₂C₆H₅ |
| | R2 = R4 = —OH |
| | R3 = —CH₂— |
| 2 | R1 = R5 = COOCH(CH₃)₂ |
| | R2 = R4 = —OH |
| | R3 = —CH₂— |
| 3 | R1 = R5 = —COOC₂H₅ |
| | R2 = R4 = —OH |
| | R3 = —CH₂— |
| 4 | R1 = R5 = —COOC₆H₁₁ |
| | R2 = R4 = —OH |
| | R3 = —CH₂— |
| 5 | R1 = R5 = COOCH₃ |
| | R2 = R4 = —OH |
| | R3 = —CH₂— |
| 6 | R1 = R5 = COOC(CH₃)₃ |
| | R2 = R4 = —OH |
| | R3 = —CH₂— |
| 7 | R1 = R5 = —CONHC₆H₅ |
| | R2 = R4 = —OH |
| | R3 = —CH₂— |
| 11 | R1 = R5 = —H |
| | R2 = R4 = —OCOC₆H₅ |
| | R3 = —CH₂— |
| 12 | R1 = R5 = —H |
| | 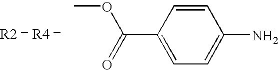 |
| | R3 = —CH₂— |
| 13 | R1 = R5 = —H |
| | R2 = R4 = OCOCH═CH₂ |
| | R3 = —CH₂—; |
| 14 | R1 = R5 = —H |
| | R2 = R4 = —OH |
| | R3 = —CH₂— |
| 15 | R1 = R5 = —COOH |
| | R2 = R4 = —OH |
| | R3 = —CH₂— |
